(19) United States
(12) Patent Application Publication
(10) Pub. No.: US 2023/0285267 A1
(43) Pub. Date: Sep. 14, 2023

(54) NOVEL HYALURONIC ACID FACIAL MASK, PREPARATION METHOD THEREFOR AND USE METHOD THEREFOR

(71) Applicant: AMOCHEM MATERIAL SCIENCE & TECHNOLOGY CO. LTD., Suzhou, Jiangsu (CN)

(72) Inventor: Xiaojiang WU, Suzhou, Jiangsu (CN)

(21) Appl. No.: 18/013,947

(22) PCT Filed: Jun. 29, 2021
(86) PCT No.: PCT/CN2021/103062

§ 371 Date: May 24, 2023

(30) Foreign Application Priority Data
Jun. 30, 2020 (CN) 202010608307.5

(51) Int. Cl. A61K 8/73; A61K 8/02; A61Q 19/08; A61K 8/86; A61K 8/88; A45D 44/00; B32B 5/02; B32B 27/34; B32B 27/12; B32B 7/02; B32B 29/02; D04H 1/728; D01D 5/00; D04H 1/42

(52) U.S. Cl. CPC A61K 8/735; A61K 8/0212; A61Q 19/08; A61K 8/86; A61K 8/88; A45D 44/002; B32B 5/022; B32B 27/34; B32B 27/12; B32B 7/02; B32B 29/02; D04H 1/728; D01D 5/0092; D04H 1/42; D01D 5/0038; D01D 5/0084; B32B 2307/7376; B32B 2307/748; B32B 2262/02; D10B 2509/00

(57) ABSTRACT

A hyaluronic acid facial mask, a preparation method therefor and a use method therefor is provided. The facial mask includes a substrate layer, a hyaluronic acid layer and a protective paper layer sequentially arranged. The hyaluronic acid layer is made of hyaluronic acid nanofibers. The hyaluronic acid nanofibers are formed by spinning a hyaluronic acid composite using an electrospinning method. The hyaluronic acid composite includes a dispersed phase and a solvent phase. The dispersed phase includes, in terms of mass percentage content, 70-95% of hyaluronic acid. The solvent phase includes water. The preparation method includes: spinning the hyaluronic acid composite on the substrate layer using an electrospinning method to form a hyaluronic acid layer, and then attaching the protective paper layer on the hyaluronic acid layer.

### CROSS-REFERENCE TO RELATED APPLICATIONS

This application claims priority to PCT Application No. PCT/CN2021/103062, having a filing date of Jun. 29, 2021, which claims priority to CN Application No. 202010608307.5, having a filing date of Jun. 30, 2020, the entire contents both of which are hereby incorporated by reference.

### FIELD OF TECHNOLOGY

The following relates to the field of hyaluronic acid facial mask technology, and in particular, relates to a novel hyaluronic acid facial mask, a preparation method therefor and a use method therefor.

### BACKGROUND

Hyaluronic acid (also known as hyaluronan) is an acidic mucopolysaccharide, which was first isolated from the vitreous humour of bovine eyes by Meyer et al, Professor of Ophthalmology at Columbia University, USA, in 1934. Hyaluronic acid, with its unique molecular structure and physicochemical properties, displays a variety of important physiological functions in the body, such as lubricating joints, regulating the permeability of blood vessel walls, regulating proteins, diffusing and transporting water-electrolyte, promoting wound healing, etc. Most importantly, hyaluronic acid has a special water retention effect and is the best moisturizing substance found in nature at present, known as the ideal natural moisturizing factor (NMF), for example: a 2% pure hyaluronic acid aqueous solution can firmly retain 98% of moisture. Hyaluronic acid is a multifunctional matrix that is widely distributed in all parts of the body, wherein the skin also contains a large amount of hyaluronic acid. Hyaluronic acid can improve the nutrient metabolism of the skin, make the skin soft, smooth, wrinkle-free, increase elasticity and prevent ageing, and is a good transdermal absorption promoter at the same time as moisturizing, which, when used in conjunction with other nutrients, can have a more desirable effect of promoting nutrient absorption, and the human skin maturation and ageing process also changes with the content of hyaluronic acid and metabolism.

At present, the more common hyaluronic acid products on the market are hyaluronic acid masks, which comprise a non-woven material and hyaluronic acid coated or impregnated on the non-woven material and a protective paper layer, however, the preparation of such masks requires the use of coating or impregnation, resulting in production efficiency is not ideal, and the amount of hyaluronic acid required is large, the substrate will absorb a large amount of hyaluronic acid, but customers can hardly make full use of all the hyaluronic acid added in the preparation process, resulting in a certain waste of resources and large production costs.

### SUMMARY

An aspect relates to a novel hyaluronic acid facial mask. The facial mask has a separate hyaluronic acid layer, which does not have the drawbacks of the conventional liquid form that needs to be attached to the substrate layer and then largely absorbed by the substrate, but can be prepared by electrospinning technology with the advantages of fast, easy, efficient and batch-produced, which greatly reduces the usage of hyaluronic acid in the production process and improves the utilization rate of hyaluronic acid, which is conducive to the full and rapid absorption of hyaluronic acid when users use hyaluronic acid facial mask. In addition, there are no preservatives, fungicides and other harmful additives.

The present disclosure also provides a preparation method for the above hyaluronic acid facial mask.

The present disclosure also provides a use method for the above hyaluronic acid facial mask.

In the present disclosure, the hyaluronic acid in the hyaluronic acid facial mask is used in its sodium salt form.

To achieve the above purpose, a technical solution employed by the present disclosure is: a hyaluronic acid facial mask comprising a substrate layer, a hyaluronic acid layer and a protective paper layer sequentially stacked, the hyaluronic acid layer being made of hyaluronic acid nanofibers, the hyaluronic acid nanofibers being formed by spinning a hyaluronic acid composite using an electrospinning method;

Wherein, the hyaluronic acid composite comprises a dispersed phase and a solvent phase; the dispersed phase comprises, in terms of mass percentage content, 70-95% of sodium hyaluronate and 5-30% of poly(ethylene oxide), the sodium hyaluronate has an average molecular weight of 1,000-100,000, and the poly(ethylene oxide) has an average molecular weight of 500,000-5,000,000; the solvent phase comprises water.

According to some aspects of the present disclosure, the sodium hyaluronate has an average molecular weight of 8,000-100,000.

In some implementations of the present disclosure, the sodium hyaluronate has an average molecular weight of 1,000-60,000.

According to some aspects of the present disclosure, the poly(ethylene oxide) has an average molecular weight of 600,000-5,000,000.

In some implementations of the present disclosure, the poly(ethylene oxide) has an average molecular weight of 500,000-2,000,000.

In the present disclosure, “the hyaluronic acid nanofibers being formed by spinning a hyaluronic acid composite using an electrospinning method” means specifically that the hyaluronic acid composite is blow spun from the spinning needle onto the substrate layer under the action of an electric field to obtain hyaluronic acid nanofibers; in turn, during the continuous blow spinning process, many hyaluronic acid nanofibers form one or several laminated layers on the substrate layer, i.e. the hyaluronic acid layer is obtained; wherein, in some implementations of the present disclosure, the operating parameters of the electrospinning method are: electric field voltage 20 to 200 KV, spinning distance (distance between the spinning needle and the substrate layer) 5 to 30 cm, spinning current (current in the circuit during the spinning process) 0.01 to 1 mA. According to some aspects of the present disclosure, the operating parameters of the electrospinning method are: electric field voltage 36 to 70 KV, spinning distance 15 to 25 cm, spinning current 0.23 to 0.42 mA.

In the present disclosure, the “electrospinning method” itself is an existing technology, but this application innovatively proposes its application in the preparation of facial masks, in particular hyaluronic acid facial masks, specifically in the preparation of hyaluronic acid layers, which does not have the drawbacks of the conventional liquid form that needs to be attached to the substrate layer and then largely absorbed by the substrate (the conventional operation is coating or impregnation), while also is able to have the process advantages of fast, easy, efficient and batch-produced; in particular, the present disclosure innovatively provides a specific hyaluronic acid composite that allows it to be applied to the “electrospinning method” and to obtain the desired hyaluronic acid layer, overcoming the problems of the conventional art.

According to some aspects of the present disclosure, in the hyaluronic acid composite, in terms of mass percentage content, the dispersed phase accounts for 5-25%, and the solvent phase accounts for 75-95%.

According to some specific aspects of the present disclosure, the hyaluronic acid composite is prepared by mixing the dispersed phase and the solvent phase evenly.

In the present disclosure, the substrate layer provides support for the hyaluronic acid layer and the protective paper layer, and can be made from hollow mesh fabric, non-woven fabric, etc., and can be made of polypropylene, nylon, etc.; preferably, the substrate layer is a nylon-based mesh substrate layer with a hollow structure.

According to some aspects of the present disclosure, the hyaluronic acid nanofibers have an average diameter of 50-500 nm.

According to some aspects of the present disclosure, the hyaluronic acid layer has a thickness of 0.005-0.1 mm. In some implementations of the present disclosure, the hyaluronic acid layer has a thickness of 0.003-0.009 mm.

According to some aspects of the present disclosure, the thickness of the hyaluronic acid layer, the thickness of the substrate layer and the thickness of the protective paper layer is 0.8-16:1:1.5-3. Further, the thickness of the hyaluronic acid layer, the thickness of the substrate layer and the thickness of the protective paper layer is 1.2-8.5:1:1.5-1.8.

According to some specific aspects of the present disclosure, the protective paper layer is used to protect the hyaluronic acid layer from damage, and can be food-grade protective paper, PE film, non-woven fabric, etc., which can be removed at the time of use by the customer.

Yet another technical solution provided by the present disclosure is: a preparation method for a hyaluronic acid facial mask mentioned above, which comprises: spinning the hyaluronic acid composite on the substrate layer using an electrospinning method to form the hyaluronic acid layer, and then attaching the protective paper layer on the hyaluronic acid layer.

In the present disclosure, the solvent phase in the hyaluronic acid composite is almost evaporated during the electrospinning process.

Still another technical solution provided by the present disclosure is: a use method for a hyaluronic acid facial mask mentioned above, which comprises: during use, applying water to a part to be applied, and then directly attaching the hyaluronic acid layer of the facial mask from which the protective paper layer has been removed to the part to be applied, the part to be applied comprises the face.

Due to the use of the above technical solutions, the present disclosure has the following advantages over the conventional art:

The present disclosure innovatively provides hyaluronic acid nanofibers made of hyaluronic acid nanofibers, which are based on the hyaluronic acid composite innovatively provided by the present disclosure, which enables smaller molecular-weight hyaluronic acid or sodium hyaluronate to be adapted to electrospinning technology to make hyaluronic acid nanofibers, which in turn ensures that the hyaluronic acid nanofibers are then used to make a separate layer of hyaluronic acid. The separate layer of hyaluronic acid not only avoids the existing drawbacks of hyaluronic acid adhering to the substrate layer and being largely absorbed by the substrate layer, and the addition of harmful substances such as preservatives and antibacterial agents, but also improves the absorption efficiency and effectiveness of hyaluronic acid in the part to be applied, thus it can be achieved that, on the one hand, the preparation process of the facial mask of the present disclosure can have the advantages of fast, easy, efficient and batch-produced due to the hyaluronic acid layer can be prepared by electrospinning technology, on the other hand, the amount of hyaluronic acid used is reduced and the utilization rate of hyaluronic acid is increased, which saves costs and enhances the consumer experience; at the same time, the hyaluronic acid facial mask of the present disclosure has better environmental adaptability (less moisture absorption, less deformation and curling) and experience of use.

### BRIEF DESCRIPTION

Some of the embodiments will be described in detail, with references to the following Figures, wherein like designations denote like members, wherein:

FIG. 1 is a scanning electron microscope image of Embodiment 1;

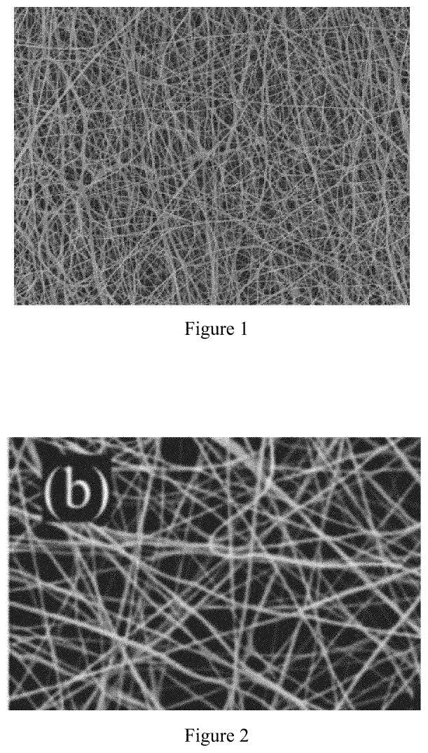

FIG. 2 is a scanning electron microscope image of Embodiment 2;

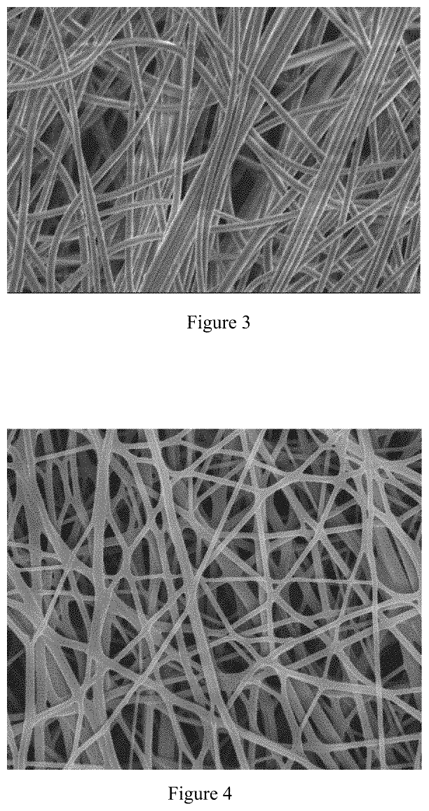

FIG. 3 is a scanning electron microscope image of Embodiment 3; and

FIG. 4 is a scanning electron microscope image of Embodiment 4.

### DETAILED DESCRIPTION

The specific embodiments below are combined to further explain the present disclosure in detail. It should be understood that, those embodiments are to explain the basic principle, major features and advantages of the present disclosure, and the present disclosure is not limited by the scope of the following embodiments. The implementation conditions employed by the embodiments may be further adjusted according to particular requirements, and undefined implementation conditions usually are conditions in conventional experiments.

In the following embodiments, unless otherwise specified, all raw materials are basically commercially available or prepared by conventional methods in the field.

### Embodiment 1

A hyaluronic acid facial mask provided by this embodiment comprises a substrate layer made of nylon-based mesh substrate layer with a hollow structure, a hyaluronic acid layer and a protective paper layer sequentially arranged, the hyaluronic acid layer is made of hyaluronic acid nanofibers, the hyaluronic acid nanofibers are formed by spinning a hyaluronic acid composite using an electrospinning method;

Wherein, the formulation of the hyaluronic acid composite and the amount of the ingredients are shown in Table 1, and the hyaluronic acid composite was prepared by mixing the dispersed phase and the solvent phase evenly.

TABLE 1

| | | Embodiment 1 |
|---|---|---|
| | | (Parts) |
| Dispersed | Sodium hyaluronate (with an average | 4.0% |
| phase | molecular weight of 100,000, purchased | |
| | from Qingdao Amochem) | |
| | Poly(ethylene oxide) (with an average | 1.0% |
| | molecular weight of 5,000,000, purchased | |
| | from Qingdao Amochem) | |
| Solvent | Water | 95% |
| phase | | |

The preparation method was: spinning the hyaluronic acid composite on the substrate layer using an electrospinning method to form the hyaluronic acid layer, and then attaching the protective paper layer on the hyaluronic acid layer; wherein, the operating parameters of the electrospinning method were: electric field voltage 36 KV, spinning distance 15 cm, and spinning current 0.23 mA; the thickness of the hyaluronic acid layer was about 0.03 mm, and the ratio of the thickness of the hyaluronic acid layer, the thickness of the substrate layer and the thickness of the protective paper layer was about 1.2:1:1.8.

### Embodiment 2

A hyaluronic acid facial mask provided by this embodiment comprises a substrate layer made of nylon-based mesh substrate layer with a hollow structure, a hyaluronic acid layer and a protective paper layer sequentially arranged, the hyaluronic acid layer is made of hyaluronic acid nanofibers, the hyaluronic acid nanofibers are formed by spinning a hyaluronic acid composite using an electrospinning method;

Wherein, the formulation of the hyaluronic acid composite and the amount of the ingredients are shown in Table 2, and the hyaluronic acid composite was prepared by mixing the dispersed phase and the solvent phase evenly.

TABLE 2

| | | Embodiment 2 |
|---|---|---|
| | | (Parts) |
| Dispersed | Sodium hyaluronate (with an average | 9% |
| phase | molecular weight of 60,000, purchased | |
| | from Qingdao Amochem) | |
| | Poly(ethylene oxide) (with an average | 1.0% |
| | molecular weight of 2,000,000, purchased | |
| | from Qingdao Amochem) | |
| Solvent | Water | 90% |
| phase | | |

The preparation method was: spinning the hyaluronic acid composite on the substrate layer using an electrospinning method to form the hyaluronic acid layer, and then attaching the protective paper layer on the hyaluronic acid layer; wherein, the operating parameters of the electrospinning method were: electric field voltage 55 KV, spinning distance 17 cm, and spinning current 0.31 mA; the thickness of the hyaluronic acid layer was about 0.05 mm, and the ratio of the thickness of the hyaluronic acid layer, the thickness of the substrate layer and the thickness of the protective paper layer was about 2.5:1:1.8.

### Embodiment 3

A hyaluronic acid facial mask provided by this embodiment comprises a substrate layer made of nylon-based mesh substrate layer with a hollow structure, a hyaluronic acid layer and a protective paper layer sequentially arranged, the hyaluronic acid layer is made of hyaluronic acid nanofibers, the hyaluronic acid nanofibers are formed by spinning a hyaluronic acid composite using an electrospinning method;

Wherein, the formulation of the hyaluronic acid composite and the amount of the ingredients are shown in Table 3, and the hyaluronic acid composite was prepared by mixing the dispersed phase and the solvent phase evenly.

TABLE 3

| | | Embodiment 3 |
|---|---|---|
| | | (Parts) |
| Dispersed | Sodium hyaluronate (with an average | 10.5% |
| phase | molecular weight of 25,000, purchased | |
| | from Qingdao Amochem) | |
| | Poly(ethylene oxide) (with an average | 1.5% |
| | molecular weight of 1,000,000, purchased | |
| | from Qingdao Amochem) | |
| Solvent | Water | 88% |
| phase | | |

The preparation method was: spinning the hyaluronic acid composite on the substrate layer using an electrospinning method to form the hyaluronic acid layer, and then attaching the protective paper layer on the hyaluronic acid layer; wherein, the operating parameters of the electrospinning method were: electric field voltage 68 KV, spinning distance 25 cm, and spinning current 0.36 mA; the thickness of the hyaluronic acid layer was about 0.07 mm, and the ratio of the thickness of the hyaluronic acid layer, the thickness of the substrate layer and the thickness of the protective paper layer was about 3.1:1:1.8.

### Embodiment 4

A hyaluronic acid facial mask provided by this embodiment comprises a substrate layer made of nylon-based mesh substrate layer with a hollow structure, a hyaluronic acid layer and a protective paper layer sequentially arranged, the hyaluronic acid layer is made of hyaluronic acid nanofibers, the hyaluronic acid nanofibers are formed by spinning a hyaluronic acid composite using an electrospinning method;

Wherein, the formulation of the hyaluronic acid composite and the amount of the ingredients are shown in Table 4, and the hyaluronic acid composite was prepared by mixing the dispersed phase and the solvent phase evenly.

TABLE 4

| | | Embodiment 4 |
|---|---|---|
| | | (Parts) |
| Dispersed | Sodium hyaluronate (with an average | 21% |
| phase | molecular weight of 8,000, purchased | |
| | from Qingdao Amochem) | |
| | Poly(ethylene oxide) (with an average | 3% |
| | molecular weight of 600,000, purchased | |
| | from Qingdao Amochem) | |
| Solvent | Water | 76% |
| phase | | |

The preparation method was: spinning the hyaluronic acid composite on the substrate layer using an electrospinning method to form the hyaluronic acid layer, and then attaching the protective paper layer on the hyaluronic acid layer; wherein, the operating parameters of the electrospinning method were: electric field voltage 70 KV, spinning distance 20 cm, and spinning current 0.42 mA; the thickness of the hyaluronic acid layer was about 0.09 mm, and the ratio of the thickness of the hyaluronic acid layer, the thickness of the substrate layer and the thickness of the protective paper layer was about 8.5:1:1.5.

Comparison 1

This example was basically the same as in Embodiment 1, by differing in that, only sodium hyaluronate was used as the dispersed phase, without the addition of poly(ethylene oxide).

Effectiveness Evaluation

1. Scanning Electron Microscope

As shown in FIG. 1, the spun yarn in Embodiment 1 is uniform and dense;

As shown in FIG. 2, the spun yarn in Embodiment 2 is also uniform and dense (at a different magnification than in Embodiment 1);

As shown in FIG. 3, the spun yarn in Embodiment 3 is uniform, with no interlacing of the fiber layers;

As shown in FIG. 4, the spun yarn in Embodiment 4 is uniform and dense, with smaller diameter;

Comparison 1 cannot be spun, the liquid was squirted straight out.

2. Yarn Surface Condition

| | |
|---|---|
| Embodiment 1 | The surface of the yarn layer was uniformly |
| | colored with a few micropores |
| Embodiment 2 | The surface of the yarn layer was uniform |
| | with very few micropores |
| Embodiment 3 | The surface of the yarn layer was smooth |
| | and uniformly colored |
| Embodiment 4 | The surface of the yarn layer was uniformly |
| | colored, smooth and delicate |
| Comparison 1 | Unable to spin |

3. Curling of Mask and Mesh Substrate Layer Under High Humidity Conditions

With the rainy weather in the southern part of the country, the air was very humid, on the day of the test the humidity was around 90%, while several samples were opened and laid flat on a table to observe the dissolution and curling of the mask and mesh substrate layers.

| | |
|---|---|
| Embodiment 1 | The white mask had no change in color and slightly |
| | curled at both chins after 30 min |
| Embodiment 2 | The white mask was translucent in color and slightly |
| | curled at both chins after 30 min |
| Embodiment 3 | The white mask had no change in color and no curling |
| | at several incisions after 30 min |
| Embodiment 4 | The white mask had no change in color and no curling |
| | at several incisions after 30 min |
| Comparison 1 | No finished product to be tested |

Note:
Under high humidity, moisture in the air will be absorbed into the hyaluronic acid layer, showing a slightly soluble state, resulting in the color of the mask layer becoming transparent;

4. Use of Mask

| | Effects of | |
|---|---|---|
| | water | |
| Cases | supplement | Feelings |
| Embodiment 1 | 8 | The hyaluronic acid layer was well distributed, the |
| | | rapid penetration and disappearance of hyaluronic acid |
| | | was observed when applied to the face, and after |
| | | application, the face was slightly tight, and the face |
| | | tightness was improved after patting with water twice; |
| | | a noticeable effect of water supplement on the face |
| | | was observed after waking up in the morning. |
| Embodiment 2 | 9 | The hyaluronic acid layer was well distributed, the |
| | | rapid penetration and disappearance of hyaluronic acid |
| | | was observed when applied to the face, and after |
| | | application, the face was tight, and felt sticky after |
| | | patting with water, and the tight and sticky feeling |
| | | disappeared after 4 patting with water. |
| Embodiment 3 | 9 | The hyaluronic acid layer was soft, and can be seen |
| | | through the gauze, the rapid disappearance of |
| | | hyaluronic acid was observed when applied to the |
| | | face, the experience was good, the face was tight after |
| | | application, and felt fresh and non-greasy after 4 |
| | | patting with water, and the skin was not oily when |
| | | wake up the next morning. |
| Embodiment 4 | 10 | The skin was tight after application, after removing |
| | | the mesh cloth, a stickiness on the surface of the skin |
| | | can be felt after patting with the hand to replenish |
| | | moisture, and the skin had an obvious improvement in |
| | | skin glossiness! |
| Comparison 1 | / | No product experience |

Note:
Rating: 1-10, with 1 being the worst and 10 being the best.

The embodiments described above are only for illustrating the technical concepts and features of the present disclosure, and are intended to make those skilled in the art being able to understand the present disclosure and thereby implement it, and should not be concluded to limit the protective scope of this disclosure. Any equivalent variations or modifications according to the spirit of the present disclosure should be covered by the protective scope of the present disclosure.

The endpoints and any values of the ranges disclosed herein are not limited to the precise ranges or values, and these ranges or values should be understood to include values close to these ranges or values. For ranges of value, between the end values of each range, between the end values of each range and individual point values, and between individual point values can be combined with each other to obtain one or more new ranges of value, and these ranges of value should be considered as specifically disclosed herein.

Although the present invention has been disclosed in the form of embodiments and variations thereon, it will be understood that numerous additional modifications and variations could be made thereto without departing from the scope of the invention.

For the sake of clarity, it is to be understood that the use of ‘a’ or ‘an’ throughout this application does not exclude a plurality, and ‘comprising’ does not exclude other steps or elements.

1. A hyaluronic acid facial mask, wherein the hyaluronic acid facial mask comprises:
a substrate layer, a hyaluronic acid layer, and a protective paper layer sequentially stacked, the hyaluronic acid layer is made of hyaluronic acid nanofibers, the hyaluronic acid nanofibers are formed by spinning a hyaluronic acid composite using an electrospinning method; wherein the hyaluronic acid composite comprises a dispersed phase and a solvent phase, the dispersed phase comprises, in terms of mass percentage content, 70-95% of sodium hyaluronate and 5-30% of poly(ethylene oxide), the sodium hyaluronate has an average molecular weight of 1,000-100,000, and the poly(ethylene oxide) has an average molecular weight of 500,000-5,000,000, and the solvent phase comprises water.

2. The hyaluronic acid facial mask according to claim 1, wherein the sodium hyaluronate has an average molecular weight of 8,000-100,000.

3. The hyaluronic acid facial mask according to claim 1, wherein the poly(ethylene oxide) has an average molecular weight of 600,000-5,000,000.

4. The hyaluronic acid facial mask according to claim 1, wherein the operating parameters of the electrospinning method are: electric field voltage 20 to 200 KV, spinning distance 5 to 30 cm, spinning current 0.01 to 1 mA.

5. The hyaluronic acid facial mask according to claim 4, wherein operating parameters of the electrospinning method are: electric field voltage 36 to 70 KV, spinning distance 15 to 25 cm, and spinning current 0.23 to 0.42 mA.

6. The hyaluronic acid facial mask according to claim 1, wherein in the hyaluronic acid composite, in terms of mass percentage content, the dispersed phase accounts for 5-25%, and the solvent phase accounts for 75-95%.

7. The hyaluronic acid facial mask according to claim 1, wherein the hyaluronic acid composite is consisted of a dispersed phase and a solvent phase, the dispersed phase is consisted of sodium hyaluronate and poly(ethylene oxide), and the solvent phase is water.

8. The hyaluronic acid facial mask according to claim 1, wherein the substrate layer is a nylon-based substrate layer.

9. The hyaluronic acid facial mask according to claim 8, wherein the nylon-based substrate layer is a nylon-based mesh substrate layer with a hollow structure.

10. The hyaluronic acid facial mask according to claim 1, wherein the hyaluronic acid nanofibers have an average diameter of 50 to 500 nm.

11. The hyaluronic acid facial mask according to claim 1, wherein the hyaluronic acid layer has a thickness of 0.005-0.1 mm.

12. The hyaluronic acid facial mask according to claim 1, wherein the hyaluronic acid layer has a thickness of 0.003-0.009 mm.

13. The hyaluronic acid facial mask according to claim 1, wherein a thickness of the hyaluronic acid layer, a thickness of the substrate layer, and a thickness of the protective paper layer is 0.8-16:1:1.5-3.

14. The hyaluronic acid facial mask according to claim 1, wherein a thickness of the hyaluronic acid layer, a thickness of the substrate layer, and a thickness of the protective paper layer is 1.2-8.5:1:1.5-1.8.

15. A preparation method for a hyaluronic acid facial mask according to claim 1, wherein the preparation method comprises: spinning the hyaluronic acid composite on the substrate layer using an electrospinning method to form the hyaluronic acid layer, and then attaching the protective paper layer on the hyaluronic acid layer.

16. A use method for a hyaluronic acid facial mask according to claim 1, wherein the method further comprising: during use, applying water to a part to be applied, and then directly attaching the hyaluronic acid layer of the hyaluronic acid facial mask from which the protective paper layer has been removed to the part to be applied, wherein the part to be applied comprises the face.

17. A use method for a hyaluronic acid facial mask according to claim 16, wherein the method further comprising: after attaching the hyaluronic acid layer of the hyaluronic acid facial mask to the part to be applied, applying water at least once to the part to be applied with the hyaluronic acid layer.

18. A hyaluronic acid facial mask according to claim 11, wherein the hyaluronic acid layer has a thickness of 0.03-0.09 mm.
